(19) United States
(12) Patent Application Publication
(10) Pub. No.: US 2005/0236127 A1
(43) Pub. Date: Oct. 27, 2005

(54) Starch compositions and methods of making starch compositions

(76) Inventors: David J. Neivandt, Bangor, ME (US); Joseph M. Genco, Orono, ME (US); Mark A. Paradis, Old Town, ME (US)

Correspondence Address: MACMILLAN SOBANSKI & TODD, LLC;ONE MARITIME PLAZA FOURTH FLOOR, 720 WATER STREET, TOLEDO, OH, 43604-1619, US

(21) Appl. No.: 11/171,667

(22) Filed: Jun. 30, 2005

Related U.S. Application Data
(62) Division of application No. 10/788,839, filed on Feb. 27, 2004.
(60) Provisional application No. 60/450,277, filed on Feb. 27, 2003.

(51) Int. Cl. D21H 17/29; C08B 30/12; C13J 1/06; C07H 1/00; C08B 31/00

(52) U.S. Cl. 162/175; 127/32; 127/46.1; 536/1.11; 536/102; 536/106

(57) ABSTRACT

A starch composition is made by cooking a starch and combining the cooked starch with a polymer, the polymer containing anionic groups or potential anionic groups. Another starch composition is made by combining a starch with a polymer, the polymer containing anionic groups or potential anionic groups, and cooking the combined starch and polymer composition. A dry starch composition, suitable for forming an additive for a paper furnish, includes a starch and a polymer containing anionic groups or potential anionic groups. Methods of making the starch composition are also disclosed.

### CROSS-REFERENCE TO RELATED APPLICATION

This application claims the benefit of U.S. provisional application Ser. No. 60/450,277, filed on Feb. 27, 2003.

### TECHNICAL FIELD

This invention relates in general to modified starch compositions of the type suitable for use as clarifying aids for removing solids and other suspended materials from aqueous dispersions, and in particular for use as retention aids in the manufacture of paper. The invention also relates to methods for manufacturing such modified starch compositions.

### BACKGROUND OF THE INVENTION

The manufacture of paper involves forming an aqueous dispersion or “furnish” of cellulosic fibers, filler particles and potentially other materials, and then draining the furnish over a wire mesh to form a sheet. Various materials have been added to the furnish to improve retention on the sheet of the filler particles and short cellulosic fibers. For example, modified starches are frequently used for this purpose.

U.S. Pat. Nos. 5,859,128 and 6,048,929 (Moffett, R.) disclose a modifed starch for use as a retention aid in a paper furnish. The modified starch is prepared by cooking, under alkaline conditions, at least one amphoteric or cationic starch with at least one polyacrylamide. U.S. Pat. Nos. 5,482,693 (Rushmere, J., Moffett, R.), 5,176,891 (Rushmere, J.) and 4,954,220 (Rushmere, J.) present a process for producing water-soluble polyparticulate polyaluminosilicate microgels.

U.S. Pat. No. 5,178,730 (Bixler, H., Peats, S.) discloses that an improvement in retention can be achieved by adding a medium/high molecular weight cationic polymer or by adding a natural hectorite to the furnish.

U.S. Pat. No. 4,643,801 (Johnson, K.) discloses a binder comprising a cationic starch in combination with an anionic high molecular weight polymer and a dispersed silica to improve retention. Similarly, U.S. Pat. No. 4,388,150 (Sunden, O., et al.) discloses that an improvement can be found with the use of colloidal silicic acid and cationic starch.

U.S. Pat. No. 4,066,495 (Voight, J.; Pender H.) presents a method of adding a cationic starch and an anionic polyacrylamide polymer to the pulp in a papermaking process to improve retention.

U.S. Pat. No. 5,294,301 (Kumar, et al.) discloses a process for the manufacture of paper from an aqueous pulp furnish, the improvement comprising adding to the aqueous pulp furnish at least about 0.1% based on the weight of the pulp, of at least one graft copolymer of starch, where the graft copolymer has an add-on amount of polymethacrylic or polyacrylic acid.

### SUMMARY OF THE INVENTION

This invention relates to compositions obtained by cooking starch and combining the starch, before or after the cooking, with a polymer containing anionic groups, such as acidic groups or salts of acidic groups. The resulting modified starch compositions can be used as clarifying aids for removing solids and other suspended materials from aqueous dispersions, and in particular as retention aids in the manufacture of paper.

According to this invention there is also provided a starch composition made by cooking a starch and combining the cooked starch with a polymer, the polymer containing anionic groups or potential anionic groups.

According to this invention there is also provided a starch composition made by combining a starch with a polymer, the polymer containing anionic groups or potential anionic groups, and cooking the combined starch and polymer composition.

According to this invention there is also provided a dry starch composition suitable for forming an additive for a paper furnish, the starch composition comprising a starch and a polymer containing anionic groups or potential anionic groups.

According to this invention there is also provided a method of making a starch composition containing a polymer, the method comprising combining a starch and a polymer to form a starch composition, cooking the starch composition at a pH less than the pKa of the polymer to form a cooked starch composition, and then raising the pH of the cooked starch composition above the pKa of the polymer.

According to this invention there is also provided a method of making a composition suitable for adding to a paper furnish, the method comprising cooking a starch, combining the cooked starch and a polymer to form a combination having a pH lower than the pKa of the polymer, and then raising the pH of the combined starch and polymer composition to a level greater than the pKa of the polymer.

According to this invention there is also provided a method of making a composition suitable for adding to a paper furnish, the method comprising combining a starch and a polymer, and cooking the combined starch and polymer at a pH greater than the pKa of the polymer.

According to this invention there is also provided a method of making a composition suitable for adding to a paper furnish, the method comprising cooking a starch, and then combining the cooked starch and a polymer, wherein the pH of the cooked starch and polymer composition is greater than the pKa of the polymer.

### DETAILED DESCRIPTION OF THE INVENTION

The invention relates to solution clarification and in particular, the retention of cellulosic species, inorganic fillers, and hydrophobic suspensions such as pitch, fatty acids, sizing agents, organic fluorocarbons and other materials used in the papermaking process. The prior art has typically centered on the addition of cationic retention aids to the papermaking furnish.

According to one embodiment of the invention, a starch, preferably having a degree of substitution between about 0.01 to 0.30, is cooked at a temperature above about 60° C. in an aqueous solution for a time effective to modify the starch. Preferably, the starch is amphoteric or cationic, and most preferably the starch is cationic. Care should be taken to remain above the pH level at which starch degradation via hydrolysis begins. The starch is combined with a polymer containing anionic groups, such as acidic groups, or salts of acidic groups, or combinations of acidic groups and salts of acidic groups, after the cook. Alternatively, the polymer can contain potential anionic groups, which are groups that can be converted into anionic groups. Examples of potential anionic groups include, but are not limited to, amide, ester, nitrile, acyl halide, aryl halide, alkyl halide, acid halide, aldehyde, alcohol, alkylbenzene, ketone and anhydride groups. Methods of converting a potential. anionic group into an anionic group include, but are not limited to, dissolution, raising the pH of the composition, heating the composition, varying the salt concentration of the composition, and irradiating the composition. Gelation may be induced directly upon addition through selection of a pH greater than the pKa of the polymer added. Alternatively the polymer may be added at a pH lower than its own pKa to facilitate mixing prior to raising the pH above the pKa of the added polymer, hence inducing gelation. The pKa of a polymer may be considered to be the pH of the polymer solution at and below which the acidic groups of the polymer are predominantly protonated and consequently the polymer is essentially neutralized. The pKa of an acid can be expressed as the negative logarithm to the base 10 of the dissociation constant Ka of the acid, according to the equation pKa=−log 10 Ka.

Although the polymer preferably contains acidic groups or salts of acidic groups, it may also contain cationic groups and therefore potentially the polymer will be amphoteric. Examples of potentially suitable cationic groups are quaternary ammonium and tertiary amines. Other cationic groups may also be suitable. Examples of acidic functionalities which the polymer may contain include but are not limited to carboxylic, sulfuric, sulfonic, phosphoric, phosphuric, phosphonic and nitric acidic groups and salts of these groups. Typical examples of polymers suitable for use with the invention include, but are not limited to, polyacrylates, such as polyacrylic acid and polymethacrylic acid, polysulfonates, such as polystryrenesulfonic acid, polyphosphates, synthetic polymers, and natural polymers or modified natural polymers, such as carboxymethylcellulose, guar and xanthan gums, and copolymers of polyacrylic acid and polyacrylamide. The polymer may be a homopolymer or a copolymer.

Alternatively the starch may be cooked in the presence of a polymer containing acidic groups or salts of acidic groups or combinations of acidic groups and salts of acidic groups. The polymer may also contain cationic groups and therefore potentially be amphoteric. Gelation may be induced during cooking through selection of a pH greater than the pKa of the polymer added. Alternatively the pH of the cook may be lower than pKa of the polymer to facilitate mixing, with the pH subsequently being raised above the polymer's pKa to induce gelation.

The anionic, amphoteric, non-ionic, or cationic starch may be any of those previously used in papermaking, or other suitable starches. Cationic starch may be derived from any of the common starch producing materials such as corn starch, potato starch, tapioca starch, and wheat starch. Cationization can be achieved by any suitable procedure, such as by the addition of 3-chloro-2-hydroxypropyltrimethylammonium chloride, to obtain cationic starches with various degrees of nitrogen substitution. The degree of cationic substitution on the starches (wt. % nitrogen/starch) can range from about 0.01 to about 0.30, preferably between 0.02 and 0.15. Naturally occurring amphoteric starches, such as potato starch, or synthetic amphoteric starches, also may be selected.

It may be convenient for the cooking to be accomplished using a starch cooker at a paper mill. A batch cooker or continuous cooker, such as a jet cooker, may be selected. The solids content during cooking is preferably less than about 15%, but higher solids concentrations may be used if adequate mixing can be accomplished. Batch cooking generally is conducted at a temperature within the range of from about 60° C. to about 100° C., and preferably at atmospheric pressure. Batch cooking at greater than atmospheric pressure can be practiced, thus enabling higher cooking temperatures. Continuous jet cooking typically is conducted at temperatures within the range of from about 60° C. to about 130° C., and preferably at 1 atmosphere and higher pressures. Higher cooking temperatures can be used if decomposition of the starch is prevented.

Cooking time should be sufficient to allow the starch to gelatinize. The selected cooking time will vary with the selected ingredients, cooking equipment and temperature, but typically will be a time within the range of from less than a second to about an hour. Longer cooking times are generally required at lower cooking temperatures. Cooking pH may be adjusted with conventional acids, bases, or salts of acids or bases, such as sulfuric acid, nitric acid, hydrochloric acid, carbon dioxide producing carbonic acid, sodium hydroxide, and potassium hydroxide. Aluminum compounds, such alum, polyaluminum chlorides, and aluminates, such as sodium aluminate and potassium aluminate, can be used to change pH and boost retention performance. Surprisingly, retention performance is thereby improved even in acid paper furnishes. Further, it has been found that inclusion of the alkaline aluminum compound in the cooking solution results in a modified starch that permits use of a non-aluminized microparticulate retention aid in acidic paper furnishes, to further improve retention performance, whereas these non-aluminized retention aids typically do not perform well in acidic paper furnishes.

The modified starch composition may be added to any suitable paper furnish as a retention aid to improve the retention of fines, fillers and other suspended material. The paper furnish may contain a variety of wood pulp and inorganic fillers, and typically has a pH within the range of from about 3 to about 10. Thus chemical, mechanical, chemi-mechanical and semi-chemical pulps may be used together with clays, precipitated or ground calcium carbonate, titanium dioxide, silica, talc and other inorganic fillers if desired. Such fillers typically are used at the 5% to 30% loading level, as a weight percent of the total paper weight, but may reach levels as high as 35%, or higher, for some specialty applications.

One particular embodiment of the invention comprises a dry starch composition suitable for forming an additive for a paper furnish. The dry starch composition includes a starch and a polymer containing anionic groups, such as acidic groups or salts of acidic groups. The dry starch composition can include a polymer containing anionic groups or potential anionic groups, such as any one or more of the acidic groups or salts of acidic groups disclosed above, and the polymer can be any one or more of the various polymers disclosed above. The dry starch composition of the invention can be prepared, transported and stored as a dry mixture. Any suitable liquid, such as water, can be added to the dry starch composition to make a wet solution suitable for cooking and adding as an additive to a paper furnish.

Particularly advantageous results are obtained when the dry starch composition, the wet starch composition, or the paper furnish also contains an anionic inorganic colloid. Thus the composition may contain, for example, montmorillonite, bentonite, silica sols, aluminum modified silica sols, aluminum silicate sols, polysilicic acid, polysilicate microgels and polyaluminosilicate microgels, separately or in combination.

The dry starch composition, the wet starch composition, or the paper furnish also may contain other typical additives, such as internal sizing agents, wet and dry strength agents, biocides, aluminum compounds (such as alum, aluminates, polyaluminum chlorides, etc.), cationic polymers (retention aids and flocculants), anionic polymers, and/or separate additions of starch. Aluminum compounds in particular have been found to boost retention performance of the invention.

As mentioned above, the method of making a starch composition can be carried out by combining the starch and polymer. to form a starch composition, cooking the starch composition at a pH less than the pKa of the polymer to form a cooked starch composition, and then raising the pH of the cooked starch composition above the pKa of the polymer. Also, the method can be carried out by cooking a starch, combining the cooked starch and a polymer to form a combination having a pH lower than the pKa of the polymer, and then raising the pH of the combined starch and polymer composition to a pH to a level greater than the pKa of the polymer. Additionally, the method can be carried out by combining the starch and polymer, and cooking the combined starch and polymer at a pH greater than the pKa of the polymer. Also, the method can be accomplished by cooking a starch, and then combining the cooked starch and polymer, wherein the pH of the cooked starch and polymer composition is greater than the pKa of the polymer.

### EXAMPLES

Ash retention trials for all of the examples, unless stated otherwise, were conducted using a simulated paper furnish comprised of 85% Hammermill photo copy paper, 7.5% SAPPI Somerset Cover Gloss coated paper, 7.5% Tembec BCTMP, and 15% added virgin calcium carbonate. These components were blended together to obtain typical paper furnish properties such as zeta potential, filler content, conductivity, etc. that are often found at commercial paper mills producing alkaline wood-free coated papers. Chemical dosages are quoted in lb/ton of fiber (kg of chemical/908 kg of fiber).

To test the ash retention performance, a drainage/retention apparatus developed by the University of Maine was used. The procedures used were similar to those described in TAPPI standard T-261.

### Example 1

This example demonstrates how cooking cationic starch and an anionic polymer, carboxy-methyl-cellulose (CMC), together under neutral pH conditions yields better retention values than adding the two chemicals separately but simultaneously to a paper furnish. Four blends were prepared comprised of Stalok 160 cationic starch from A. E. Staley with varying amounts of a 3% solution of 7M CMC from Aqualon. Table 1 lists the grams of each component for the four starch/CMC blends. The blends were then each cooked in a bench-top laboratory jet cooker at a temperature and residence time of approximately 124° C. and 1 min, respectively.

TABLE 1

| | | grams of | |
|---|---|---|---|
| | dry grams of | 3% CMC | grams of |
| Starch:CMC ratio | starch | sol'n | water |
| 100:1 | 60 | 20.0 | 920.0 |
| 75:1 | 60 | 26.6 | 913.4 |
| 50:1 | 60 | 40.0 | 900.0 |
| 25:1 | 60 | 80.0 | 860.0 |

For purpose of comparison, the Stalok 160 was prepared as a 6.0% wt solution and cooked using the same procedure as described above. A 3% solution of 7M CMC was prepared by blending 18 grams of CMC and 582 grams of water under agitation for 20 minutes, and then allowed to rest for 1 hour.

For the control experiments, the starch and CMC were separately but simultaneously added to the paper furnish. The dosage of starch was 20 lb/ton (9.08 kg/908 kg) and four levels of CMC were tested including, 0.2, 0.26, 0.4 and 0.8 lb/ton (0.09, 0.12, 0.18 and 0.36 kg/908 kg). The cooked starch/CMC blends were added to the paper furnish at a rate of 20 lb/ton (9.08 kg/908 kg). The ash retention results are shown below in Table 2.

TABLE 2

| Separate Addition | | Starch/CMC Blends | |
|---|---|---|---|
| CMC dosage, | Ash Retention | Starch/CMC | Ash Retention |
| lb/ton | % | Ratio | % |
| 0.20 | 60.6 | 100:1 | 61.8 |
| 0.26 | 60.7 | 75:1 | 63.0 |
| 0.40 | 63.3 | 50:1 | 64.3 |
| 0.80 | 60.3 | 25:1 | 61.2 |

The results show that ash retention for a given CMC dosage can be improved by cooking the cationic starch and anionic CMC together, versus the separate and simultaneous addition of the starch and CMC.

### Example 2

This example demonstrates how cooking a cationic starch and anionic polymer (CMC) at a pH less than or greater than the pKa of the polymer can yield better retention values than adding the two chemicals separately but simultaneously to a paper furnish. Two starch blends were prepared by mixing 60 dry grams of Stalok 160 cationic starch from A. E. Staley with 40 grams of a 3% solution of 7M CMC from Aqualon to 900 grams of distilled water. The two batches were mixed thoroughly and the pH of one batch was adjusted to pH 7.43 with sodium hydroxide and the second batch was adjusted to 3.91 with hydrochloric acid. The blends were then each cooked in a bench-top laboratory jet cooker at a temperature and residence time of approximately 124° C. and 1 min, respectively. The pH of the acidic cooked blend was increased to a level greater than the pKa of the CMC with sodium aluminate to a pH of 7.65.

For purpose of comparison the Stalok 160 was prepared as a 6.0% wt solution and cooked using the same procedure as described above. The 3% solution of 7M CMC was prepared by blending 18 grams of CMC and 582 grams of water under agitation for 20 minutes, and then allowed to rest for 1 hour.

For the control experiment, the starch and CMC were separately but simultaneously added to the paper furnish. The dosage of starch and CMC was 20 lb/ton (9.08 kg/908 kg) and 0.4 lb/ton (0.18 kg/908 kg), respectively. The starch/CMC blends were added to the paper furnish at a rate of 20 lb/ton (9.08 kg/908 kg). The ash retention results are shown below in Table 3.

TABLE 3

| Experiment | Ash Retention |
|---|---|
| Separate addition of Starch and CMC (control) | 58.7 |
| Starch/CMC blend cooked at pH > pKa of CMC | 60.6 |
| Starch/CMC blend cooked at pH < pKa of CMC | 61.2 |

The results show that ash retention for a fixed CMC dosage can be improved by cooking the cationic starch and anionic CMC together regardless of the pH conditions.

### Example 3

This example demonstrates that the ash retention performance can be increased when a cooked starch and a polymer are blended together, wherein the pH of the cooked starch and polymer composition is greater than the pKa of the polymer over the separate and simultaneous addition of the cooked starch and polymer.

For this example, a 6% slurry of Stalok 160 from A. E. Staley was cooked in a bench-top laboratory jet cooker at a temperature and residence time of approximately 124° C. and 1 min, respectively. A 3% solution of 7M CMC was prepared by blending 18 grams of CMC and 582 grams of water under agitation for 20 minutes, and then allowed to rest for 1 hour. Four starch and CMC blends were prepared with varying levels of CMC. Table 4 lists the grams of each component for the four starch/CMC blends.

TABLE 4

| | grams | grams of | |
|---|---|---|---|
| | of 4.08% | 3% CMC | grams of |
| Starch:CMC ratio | cooked starch | sol'n | water |
| 100:1 | 122.5 | 1.67 | 75.83 |
| 75:1 | 122.5 | 2.22 | 75.28 |
| 50:1 | 122.5 | 3.33 | 74.17 |
| 25:1 | 122.5 | 6.67 | 70.83 |

For the control experiments, the starch and CMC were separately but simultaneously added to the paper furnish. The dosage of starch was 20 lb/ton (9.08 kg/907 kg) and four levels of CMC were tested including, 0.2, 0.26, 0.4 and 0.8 lb/ton (0.09, 0.12, 0.18 and 0.36 kg/908 kg). The starch/CMC blends were added to the paper furnish at a rate of 20 lb/ton (9.08 kg/908 kg). The ash retention results are shown below in Table 5.

TABLE 5

| Separate Addition | | Starch/CMC Blends | |
|---|---|---|---|
| CMC dosage, | Ash Retention | Starch/CMC | Ash Retention |
| lb/ton | % | Ratio | % |
| 0.20 | 58.4 | 100:1 | 60.3 |
| 0.26 | 58.7 | 75:1 | 60.5 |
| 0.40 | 59.7 | 50:1 | 61.2 |
| 0.80 | 59.8 | 25:1 | 60.2 |

The data in Table 5 shows that simple mixing of a cationic starch and anionic polymer prior to addition to the paper furnish yields increased ash retention performance over the separate but simultaneous addition of the separate components at a given anionic polymer level.

### Example 4

This is another example showing the synergistic effect of blending a cooked starch and a polymer together prior to the addition to the paper furnish as in the previous example, except the polymer used in this example was a amphoteric polyacrylamide containing sulfonic acid anionic groups. The cationic starch (Stalok 180 from A. E. Staley) was prepared in the same manner as the previous examples. The resulting cooked starch solids was 4.55% The amphoteric polyacrylamide was prepared at 0.1% by hydrating 1 dry gram of polymer into 999 grams of distilled water. The 0.1% PAM solution was agitated with a magnetic stirrer for 1 hour. One blend of cooked starch and hydrated PAM was made by thoroughly mixing 219.78 grams of the cooked starch (4.55% solids) and 125 grams of the 0.1% PAM.

For the control experiment, the starch and PAM were separately but simultaneously added to the paper furnish. The dosage of starch and PAM was 15 lb/ton (6.8 kg/908 kg) and 0.1875 lb/ton (0.085 kg/908 kg), respectively. The starch/PAM blend was added to the paper furnish at a rate of 15 lb/ton (6.8 kg/908 kg). The ash retention results are shown below in Table 6.

TABLE 6

| Experiment | Ash Retention |
|---|---|
| Separate addition of starch and amphoteric | 61.2 |
| PAM (control) | |
| Starch/PAM blend | 65.1 |

The results show an increase in ash retention with the use of this invention over the separate addition of the additives.

### Example 5

Example 5 demonstrates how combining a cooked starch and a polymer to form a composition having a pH lower than the pKa of the polymer, and then raising the pH of the combined starch and polymer composition to a level greater than the pKa of the polymer results in an increased ash retention performance over adding the components separately to a paper furnish. For this example, the cationic starch, Stalok 160, and the anionic polymer, 7M CMC, were prepared as described in the previous examples. Samples of cooked starch and hydrated CMC were used for the control experiments, the pH of the remaining cooked starch and the CMC were adjusted to 2.67 and 3.90, respectively, with hydrochloric acid. Four starch and CMC blends were prepared with varying levels of CMC. Once thoroughly blended, the pH of each mixture was increased to a pH greater than the pKa of the polymer using sodium aluminate. Table 7 lists the grams of each component for the four starch/CMC blends and the pH values.

TABLE 7

| | grams | grams of | | pH of mixture |
|---|---|---|---|---|
| | of 3.91% | 3% CMC | pH of | after |
| Starch:CMC ratio | cooked starch | sol'n | mixture | adjustment |
| 100:1 | 127.89 | 1.67 | 3.04 | 8.67 |
| 75:1 | 127.89 | 2.22 | 3.09 | 7.53 |
| 50:1 | 127.89 | 3.33 | 3.14 | 8.28 |
| 25:1 | 127.98 | 6.67 | 3.28 | 8.22 |

For the control experiments, the starch and CMC were separately but simultaneously added to the paper furnish. The dosage of starch was 20 lb/ton (9.08 kg/908 kg) and four levels of CMC were tested including, 0.2, 0.26, 0.4 and 0.8 lb/ton (0.09, 0.12, 0.18 and 0.36 kg/908 kg). The starch/CMC blends were added to the paper furnish at a rate of 20 lb/ton (9.08 kg/908 kg). The ash retention results are shown below in Table 8.

TABLE 8

| Separate Addition | | Starch/CMC Blends | |
|---|---|---|---|
| CMC dosage, | Ash Retention | Starch/CMC | Ash Retention |
| lb/ton | % | Ratio | % |
| 0.20 | 61.0 | 100:1 | 60.9 |
| 0.26 | 61.6 | 75:1 | 63.9 |
| 0.40 | 61.2 | 50:1 | 64.5 |
| 0.80 | 62.2 | 25:1 | 63.3 |

The data in Table 8 shows that mixing of a cationic starch and anionic polymer prior to addition to the paper furnish yields increased ash retention performance over the separate but simultaneous addition of the separate components at a given anionic polymer level.

### Example 6

Example 6 demonstrates the effect of varying the dosage of an inorganic colloid, silica, on ash retention when used with a cooked cationic starch/anionic CMC blend. For this example, the cationic starch, Stalok 160, and the anionic polymer, 7M CMC, were prepared as described in the previous examples. Samples of cooked starch and hydrated CMC were used for the control experiments, the pH of the remaining cooked starch and the CMC were adjusted to 3.45 and 4.3, respectively, with hydrochloric acid. One starch/CMC blend was prepared by mixing 150 grams of 4.1% cooked starch and 12.3 grams of 1% CMC. Once thoroughly blended, the pH of the mixture was increased to a pH greater than the pKa of the polymer using sodium aluminate. The final mixture pH was 6.77

The colloidal silica used in the experiment, Fennosil K515 from Kemira, was prepared by mixing 1.67 grams of silica (15% solids) and 998.33 grams of water to result in a 0.025% solution. The dosages of silica were 0, 0.5 and 1 lb/ton (0, 0.227 and 0.454 kg/908 kg).

For the control experiments, the starch and CMC were separately but simultaneously added to the paper furnish at a dosage of 20 lb/ton (9.08 kg/908 kg) and 0.4 lb/ton (0.18 kg/908 kg) respectively. The starch/CMC blends were added to the paper furnish at a rate of 20 lb/ton (9.08 kg/908 kg). The ash retention results are shown below in Table 9.

TABLE 9

| | Starch & CMC added | |
|---|---|---|
| Silica Dosage, lb/ton | Separately | Starch/CMC blend |
| 0 | 55.7 | 57.4 |
| 0.5 | 56.8 | 59.1 |
| 1 | 58.7 | 60.6 |

The results show that the addition of silica to the paper furnish improves the ash retention performance, with the greatest performance increase seen when used with the starch/CMC blend. Table 9 again shows that higher ash retention is achieved when blending the starch and CMC together, than by adding the starch and CMC separately and simultaneously.

### Example 7

Example 7 demonstrates that the ash retention performance of the invention can be seen when a starch and CMC blend are added to an acidic furnish containing an aluminum compound. The furnish used in this example was comprised of 33% groundwood, 25% bleached softwood kraft, 42% bleached sulphite and 25% added filler clay. The pH was adjusted to 4.44 using aluminum sulfate. For this example, the cationic starch, Stalok 160, and the anionic polymer, 7M CMC, were prepared as described in the previous examples. Samples of cooked starch and hydrated CMC were used for the control experiments. One starch/CMC blend was prepared by thoroughly mixing 347.95 grams of 4.95% cooked starch and 6.66 grams of 3% CMC.

For the control experiments, the starch and CMC were separately but simultaneously added to the paper furnish at a dosage of 20 lb/ton (9.08 kg/908 kg) and 0.25 lb/ton (0.11 kg/908 kg) respectively. The starch/CMC blends were added to the paper furnish at a rate of 20 lb/ton (9.08 kg/908 kg). The ash retention results are shown below in Table 10.

TABLE 10

| Experiment | Ash Retention |
|---|---|
| Separate addition of Starch and CMC (control) | 59.6 |
| Starch/CMC Blend | 61.0 |

The table shows the starch/CMC blend out performs the separate addition of starch and CMC in the acid furnish containing an aluminum compound.

### Example 8

Example 8 demonstrates that the ash retention performance is enhanced when a cationic starch and an polymer blend are cooked together in the presence of an aluminum compound. For this example, the anionic polymer, 7M CMC was prepared as described in the previous examples. A solution of uncooked cationic starch, Stalok 160, was made with distilled water then blended with the hydrated CMC in a dry ratio of 50:1 (starch:CMC) for a total solution solids content of 6.0% by weight. The pH of this solution was then raised to 9.0 using the aluminum compound sodium aluminate. The solution was then cooked in a jet cooker at 255° F.

For the control experiments, the starch and CMC were separately but simultaneously added to the paper furnish at a dosage of 20 lb/ton (9.08 kg/908 kg) and 0.4 lb/ton (0.18 kg/908 kg) respectively. The starch/CMC blend was added to the paper furnish at a rate of 20 lb/ton (9.08 kg/908 kg). The ash retention results are shown below in Table 11

TABLE 11

| Experiment | Ash Retention |
|---|---|
| Separate addition of Starch and CMC (control) | 53.7 |
| Starch/CMC blend containing aluminum | 55.5 |
| compound before cooking | |

The table shows the starch/CMC blend containing and aluminum compound before cooking out performed the separate addition of starch and CMC in the paper furnish.

### Example 9

Example 9 is intended to illustrate preparation of a dry product mixture and the results anticipated from addition of the resulting gel in a papermaking furnish. For example, a cationic starch such as Stalok 160 can be mixed with an anionic polymer such as 7M CMC as described previously in Example 1. Dry alum and or sodium aluminate or other pH controlling additive could be incorporated into the dry mixture to give the desired initial pH; that is, either above or below the pKa of the polymer. Preparing the starch/polymer mixtures in the dry state is advantageous because dry powdered products can be readily transported and stored. When the desired gel is to be applied on the wet end of the papermachine, the dry powder mixture would be dispersed with water and cooked in a starch cooker as illustrated in Example 1. If the mixture were cooked below the pKa of the polymer, the pH of the cooked dry mixture would be raised above the pKa of the polymer to form a gel. The pH adjustment would be made by adding either sodium hydroxide, sodium aluminate, or some other suitable base. Alternatively, the starch/polymer mixture can be cooked above the pKa of the acid directly to form the gel. It would be expected that application of the starch/polymer gels would give filler retention values similar to the data illustrated in Tables 2 and 5 when compared to addition of the components separately.

1-67. (canceled)

68. An aqueous dispersion containing suspended materials and a starch composition which functions as a clarifying aid in the aqueous dispersion, the starch composition made by cooking a starch for a time effective to gelatinize the starch, and combining the cooked starch with a polymer, the polymer containing anionic groups or potential anionic groups, the starch and the polymer being combined before adding the starch composition to the aqueous dispersion.

69. The aqueous dispersion of claim 68 in which the anionic groups or potential anionic groups are acidic groups or salts of acidic groups or a combination of acidic groups and salts of acidic groups.

70. The aqueous dispersion of claim 69 in which the acidic groups are one or more of the group consisting of carboxylic acid, sulfonic acid, phosphoric acid, salts of carboxylic acid, salts of sulfonic acid, and salts of phosphoric acid.

71. The aqueous dispersion of claim 68 in which the polymer is one or more of the group consisting of natural polymers, modified natural polymers, synthetic polymers, homopolymers of polyacrylates, homopolymers of polysulfonates, homopolymers of polyphosphates, copolymers of polyacrylates, copolymers of polysulfonates, and copolymers of polyphosphates.

72. The aqueous dispersion of claim 68 in which the polymer is one or more of the group consisting of polyacrylic acid, polymethacrylic acid, polystryrenesulfonic acid, carboxymethylcellulose, guar and xanthan gums, anionic and amphoteric starch, and copolymers of acrylic acid and acrylamide.

73. The aqueous dispersion of claim 68 in which the starch composition contains an aluminum compound prior to the cooking of the starch.

74. The aqueous dispersion of claim 68 which comprises a furnish, the suspended materials including cellulosic fibers.

75. The aqueous dispersion of claim 74 additionally comprising an inorganic colloid.

76. The aqueous dispersion of claim 74 additionally comprising an aluminum compound.

77. A dry clarifying aid product comprising a dry mixture of starch and a polymer, the polymer containing anionic groups or potential anionic groups, the dry product being in a form that is suitable to be readily transported and stored, and the dry product being suitable to be mixed with a liquid to make a wet solution suitable for cooking and then adding to an aqueous dispersion containing suspended materials to function as a clarifying aid in the aqueous dispersion.

78. The dry clarifying aid product of claim 77 additionally comprising a pH adjuster mixed with the starch and the polymer.

79. The dry clarifying aid product of claim 77 additionally comprising an aluminum compound mixed with the starch and the polymer.

80. The dry clarifying aid product of claim 77 additionally comprising an inorganic colloid mixed with the starch and the polymer.

81. The dry clarifying aid product of claim 77 in which the anionic groups or potential anionic groups are acidic groups or salts of acidic groups or a combination of acidic groups and salts of acidic groups.

82. The dry clarifying aid product of claim 81 in which the acidic groups are one or more of the group consisting of carboxylic acid, sulfonic acid, phosphoric acid, salts of carboxylic acid, salts of sulfonic acid, and salts of phosphoric acid.

83. The dry clarifying aid product of claim 77 in which the polymer is one or more of the group consisting of natural polymers, modified natural polymers, synthetic polymers, homopolymers of polyacrylates, homopolymers of polysulfonates, homopolymers of polyphosphates, copolymers of polyacrylates, copolymers of polysulfonates, and copolymers of polyphosphates.

84. The dry clarifying aid product of claim 77 in which the polymer is one or more of the group consisting of polyacrylic acid, polymethacrylic acid, polystryrenesulfonic acid, carboxymethylcellulose, guar and xanthan gums, and copolymers of acrylic acid and acrylamide.
